(19) United States
(12) Patent Application Publication
(10) Pub. No.: US 2012/0298108 A1
(43) Pub. Date: Nov. 29, 2012

(54) AUTOMATICALLY CONTROLLED VENTILATION SYSTEM

(75) Inventors: Michael Thomas Kane, Harrison City, PA (US); Benjamin Irwin Shelly, Pittsburgh, PA (US)

(73) Assignee: KONINKLIJKE PHILIPS ELECTRONICS N.V., EINDHOVEN (NL)

(21) Appl. No.: 13/521,544

(22) PCT Filed: Dec. 17, 2010
(86) PCT No.: PCT/IB2010/055922

§ 371 Date: Jul. 11, 2012

Related U.S. Application Data
(60) Provisional application No. 61/297,400, filed on Jan. 22, 2010.

(51) Int. Cl. A61M 16/00

(52) U.S. Cl. 128/204.23

(57) ABSTRACT

A system for controlling and regulating breathing gas supplied to a patient that includes a pressure generator system that provides breathing gas to a patient and a patent circuit that delivers the breathing gas to the patient. The system includes an interface device coupled to the patient circuit to communicate the flow of gas to the patient' airway. A sensor detects a parameter indicative of a flow of breathing gas delivered to the patient. A controller coupled to the sensor and the pressure generator system measures at least one characteristic of respiratory airflow of a patient and calculates a target breath-amplitude-based parameter and a time-based parameter of at least a portion of the patient's respiratory cycle to be delivered to the patient based on the at least one characteristic of respiratory airflow.

This patent application claims the priority benefit under 35 U.S.C. § 119(e) of U.S. Provisional Application No. 61/297,400 filed on Jan. 22, 2010, the contents of which are herein incorporated by reference.

The invention relates to a ventilator and a method of controlling a ventilator to supply a desired target volume of fluid, such as air or an oxygen mixture, to a patient.

Conventional ventilators may be used to deliver a fluid, such as air or an oxygen mixture, to a patient in a volume targeted ventilation mode in which the ventilator attempts to deliver to the patient, during inspiration, a preset volume of fluid. To adjust the volume of fluid delivered to the patient during inspiration to achieve this target volume during each inspiration, the ventilator adjusts the pressure of the fluid provided to the patient. For example, for a given inspiratory phase in a plurality of respiratory cycles, increasing or decreasing the pressure increases or decreases the volume of fluid delivered to the patient, respectively.

Ventilators may operate in a variety of modes. For example, volume ventilators that operate in a volume targeted ventilation (“VTV”) mode monitor the actual volume of fluid delivered to the patient during an inhalation and increase or decrease, as needed, the pressure at which the fluid is delivered to the patient to meet a target volume of fluid.

It is also known to operate a ventilator in a volume assured pressure support (“VAPS”) mode, in which the pressure is controlled by the ventilator in a manner so as to ensure that a set minimum volume is always delivered to the patient during each breath. In this mode of volume ventilation, if, during an inspiratory phase, the patient's inspiratory flow is not sufficient to provide the set volume for that breath, the ventilator transitions to a volume controlled mode of operation and increases the pressure of the fluid flow to the patient to meet this set volume. This typically occurs at the middle or near the end of the inspiratory phase when the ventilator determines that the patient's inspiratory drive will not be sufficient to achieve the set volume for that breath. Because this increase in pressure typically occurs near the end of the breath, when the patient is most likely to want to exhale, this mode of ventilation can be uncomfortable to the spontaneously breathing patient.

Some ventilators may also operate in an Average Volume Assured Pressure Support (AVAPS) mode. AVAPS delivers pressure support ventilation to a tidal volume setpoint. It adjusts the amount of pressure support slowly over several minutes to achieve a tidal volume setpoint. The tidal volume setpoint is determined manually and is inputted into the device.

Some examples of ventilation devices are continuous positive airway pressure (CPAP) devices, bi-level positive airway pressure devices, and auto servo ventilation (ASV) devices. For many obstructive sleep apnea (OSA) patients, CPAP therapy is sufficient to treat OSA by reducing the swollen nasal passages and splinting the upper airway with air pressure. Some automatically controlled CPAP devices monitor the patients' breathing activity and then select an appropriate level of CPAP pressure to maintain airway patency. A subset of the general OSA population are treated with bi-level positive airway pressure devices to provide assisted ventilation during sleep. Bi-level positive airway pressure devices may be used when splinting pressure of CPAP exceeds a comfortable level or when some degree of fixed ventilation assistance is needed.

Ventilators, such as those capable of providing a bi-level positive airway pressure, may be used to assist in ventilation of patients with obesity hypoventilation syndrome (OHS). OHS patients are those who breathe with a combination of rapid breath rates and reduced tidal volumes. These characteristics are caused by the large abdomen structure of these patients which reduces the amount of air transferred each breath. The pressure support ventilation is titrated in the lab to increase tidal volume and decrease breath rate in the sleeping patient. Without pressure support, these patients would drive 23 breaths per minute at 250 ml tidal volumes. In comparison, the optimal range for most patients is between 11-16 breaths per minute.

Another type of ventilators are auto servo ventilators that provide assistance to patients with centrally mediated breathing disorders. Assistance may be provided to patients with astable breathing control, patients with congestive heart failure, and patients with other conditions that prevent stable breathing. These devices monitor patient breathing conditions and provide ventilation assistance in the form of increased inspiratory pressure when the patient's breathing effort is less than, for example, the patient's monitored breathing efforts during previous breaths.

With current volume targeted ventilators, device settings must be determined under the direction of trained medical staff. The ventilator settings are adjusted to provide a combination of breath rate and tidal volumes that reduce the patient's work of breathing. The ideal solution is one that does not overdrive the patient into central sleep apnea and manages the patient breath rate into an optimal range. Moreover, current auto servo ventilators are not designed to provide any response to conditions marked by increased breath rate, such as tachypnea or dyspnea. Instead, they are designed to provide stable breathing at or near the patient's peak flow or tidal volume. Machine breaths are issued in the event that the patient fails to initiate a breath. The current auto servo ventilators do not recognize or attempt to optimize a patient's breathing rate by providing larger tidal volume breaths.

Some algorithms use the Otis equation to calculate the breath rate associated with the minimum work of breathing. The Otis equation shows the relationship between the resistance and compliance of the lungs and the total work of breathing. Specifically, breathing small tidal volumes with a high breath rate requires a high work of breathing due to the repetitive ventilation of upper airway resistance networks. Additionally, breathing a large tidal volume with a low breath rate requires a high work of breathing due to the work associated with the compressing of lung compliance. The Otis equation, which uses parameters reflective of the work of breathing, breath rate, minute ventilation, dead space volume, and lung compliance, is shown below:

$${V}_{A} = \frac{KDf + {K}^{′} {π}^{2} {Df}^{2} + 4 {K}^{″} {π}^{2} {D}^{2} {f}^{2}}{K - {}_{4}^{}{K}{}_{}^{″} {π}^{2} {Df}^{2}}$$

where:
“f” is the frequency of ventilation,
“Va” is the alveolar ventilation,
“K” is the lung compliance factor, and
“D” is the dead space volume.

The following equation is derived from the Otis equation and shows the breath rate associated with the minimum work of breathing:

$$f = \frac{- {K}^{′} {V}_{D} + \sqrt{{( {K}^{′} {V}_{D} )}^{2} + 4 {K}^{′} {K}^{″} {π}^{2} {V}_{AR} {V}_{D}}}{2 {π}^{2} {V}_{D} {K}^{″}}$$

where
f is the breathing frequency,
K′ is the lung elastance,
K″ is the air viscosity factor in the lungs,
V_{D} is the dead space volume, and
V_{AR} is the alveolar ventilation (V_{AR}=V_{A}/60).

The following equation has been used in some ventilator devices:

$$f = \frac{\sqrt{1 + 2 a \times RCe \times ( Min Vol - f \times Vd ) / ( Vd )} - 1}{a \times RCe}$$

where:
“a” is a factor that depends on the flow waveform (for sinusoidal flows, “a” is 2π²/60),
“RCe” is an expiratory time constant,
“MinVol” is a target minute ventilation,
“Vd” is the ventilated dead space, and
“f” is the breath rate frequency

Devices that use the above equation require an ideal body weight to be input into the device. A look up table in the device converts ideal body weight into a MinVol setpoint. Devices that use the above equation inserts MinVol, estimated dead space, and expiratory time constant into the above equation and performs iterative calculations, including feeding f back into the equation until the difference between multiple iterations is less than 0.5 breaths per minute. Once the frequency is determined, the device calculates a tidal volume for each breath Vt target by dividing the MinVol target by the ideal breath rate f. The device then controls the ventilation delivered to the patient to breath rate=f, and with sufficient pressure support to deliver Vt(target) tidal volumes. However, these devices require the ideal body weight to be predetermined and to be externally inputted into the device. As such, current devices do not automatically determine and provide an optimal target tidal volume and pressure support to be delivered to the patient based on changing breathing characteristics, or other physiological characteristics.

One aspect provides a system for controlling and regulating breathing gas supplied to a patient. The system includes a pressure generator system adapted to provide breathing gas to a patient and a patent circuit operatively coupled to the pressure generator system to deliver the flow of breathing gas to the patient. The system also includes an interface device operatively coupled to the patient circuit to communicate the flow of breathing gas to an airway of the patient. The system further includes a sensor operatively coupled to one or more of the pressure generator system, the patient circuit, and the interface device. The sensor is operable to detect one or more parameters indicative of a flow of breathing gas delivered to the patient. The system also includes a controller operatively coupled to the sensor and the pressure generator system. The controller determines at least one characteristic of respiratory airflow of a patient. The controller, based on the at least one characteristic of respiratory airflow, calculates a time-based parameter of at least a portion of the patient's respiratory cycle and also calculates a target breath-amplitude-based parameter to be delivered to the patient.

Another aspect provides a method for controlling and regulating breathing gas supplied to a patient. The method includes measuring at least one characteristic of respiratory airflow of a patient and calculating a time-based parameter of at least a portion of the patient's respiratory cycle based on the at least one characteristic of respiratory airflow of the patient. The method also includes calculating a target breath-amplitude-based parameter based on the at least one characteristic of respiratory airflow of the patient. The method further includes delivering the calculated target breath-amplitude-based parameter to the patient.

Another aspect provides a system for controlling and regulating breathing gas supplied to a patient. The system includes means for measuring at least one characteristic of respiratory airflow of a patient and means for calculating a time-based parameter of at least a portion of the patient's respiratory cycle based on the at least one characteristic of respiratory airflow of the patient. The system also includes means for calculating a target breath-amplitude-based parameter based on the at least one characteristic of respiratory airflow of the patient. The system further includes means for delivering the calculated target breath-amplitude-based parameter to the patient.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. In one embodiment of the invention, the structural components illustrated herein can be considered drawn to scale. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a schematic diagram of a pressure support system or ventilator connected to a patient via a circuit and an interface;

FIG. 2 is a waveform illustrating the inhalation and exhalation phases of patient flow;

FIG. 3 is a flow chart of the operation of the system in accordance with an embodiment;

FIG. 4 is a waveform illustrating exhalation time of patient flow; and

FIG. 5 is a waveform illustrating expiratory tail time of patient flow.

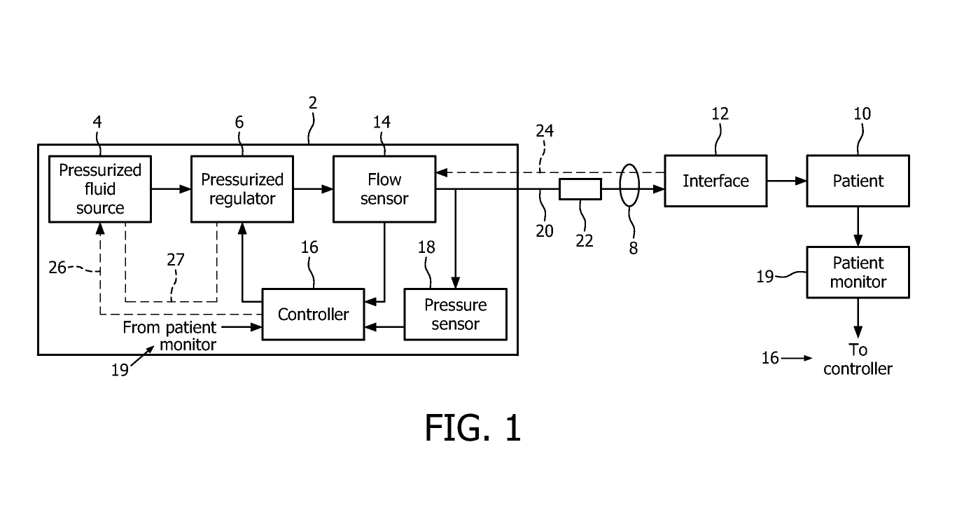

FIG. 1 illustrates an exemplary embodiment of a pressure support system or ventilator 2 according to the principles of the present invention. As used herein, the term “ventilator” refers to any device that delivers a flow of breathing gas to a patient at a variable pressure, and is not intended to be limited to a life support ventilating system.

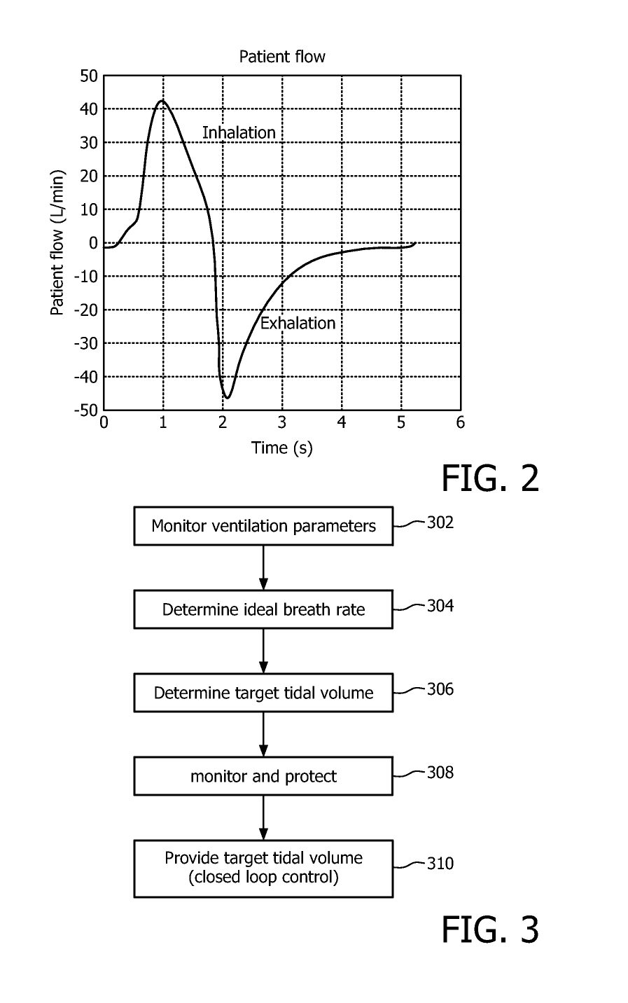

System 2 measures at least one characteristic of respiratory airflow of the patient; calculates or determines a target time-based parameter, such as an ideal breath rate, based on the at least one measured characteristic; and calculates a target breath-amplitude-based parameter, such as target tidal volume, to be delivered to the patient; and then delivers the calculated target breath-amplitude-based parameter to the patient. The relationship between flow and tidal volume is shown in FIG. 2. As shown in FIG. 2, positive flow represents the flow into the patient during inspiration. Negative flow represents the exhaled flow out of the patient. Inhaled tidal volume may be determined by integrating the positive flow. Exhaled tidal volume may be determined by integrating the negative flow.

Referring back to FIG. 1, system 2 includes a source of pressurized fluid 4 and a pressure regulator 6 connected to receive pressurized fluid from source of pressurized fluid 4. Pressure regulator 6 regulates the pressure of the pressurized fluid supplied to a patient circuit 8, which conveys the pressure regulated fluid to a patient 10 via a patient interface device 12. A sensor 14 detects a parameter associated with the fluid flow in patient circuit 8 or in interface device 12 that can be used to determine a volume of fluid supplied to the patient from pressure regulator 6 and provides to a controller 16 a signal indicative of this parameter.

In an exemplary embodiment, sensor 14 is a flow sensor that detects the flow of fluid in patient circuit 8. This flow can be used to determine the volume of fluid provided to the patient. It is to be understood, however, that the present invention contemplates using other parameters, such as the power or current provided to a blower, to determine the flow and, hence, the volume of fluid provided to a patient. A pressure sensor 18 detects the pressure of the pressurized fluid in patient circuit 8 and, more particularly, at patient interface 12, and supplies to controller 16 a signal indicative of the detected pressure. While the point at which the flow is measured by flow sensor 14 and the pressure is measured by pressure sensor 18 are illustrated as being within ventilator 2, it is to be understood that the location at which the actual flow and pressure measurements are taken be anywhere along patient circuit 8 or patient interface 12 so long as the purpose of measuring the pressure at the patient and the volume of fluid delivered to the patient can be determined.

The present invention also contemplates providing one or more patient monitors 19 to detect other physiological conditions of the patient. Such physiological conditions can be used to monitor the patient and/or control the operation of ventilator.

For example, one embodiment of the present invention contemplates that patient monitor 19 is a diaphragm electromyographic (“EMG”) detection system that detects the EMG signals produced by the diaphragm during breathing. Another example of a suitable patient monitor is an effort detector, which detects the movement of the patient's chest during respiration. Patient monitor 19 is connected to controller 16, which monitors the diaphragm EMG or effort signal supplied thereto from patient monitor 19, for example, and, in one embodiment, causes ventilator 2 to supply fluid to patient 10 during an inspiratory phase of a respiratory cycle and to terminate or reduce the supply of fluid to patient 10 during an expiratory phase. More specifically, in this embodiment of the present invention, controller 16 signals pressure regulator 6 to supply pressurized fluid to patient 10 during an inspiratory phase and to withhold or reduce the supply of pressurized fluid to patient 10 during an expiratory phase. Alternatively, as shown by dashed line 25 between controller 16 and pressurized fluid source 4, controller 16 can control pressurized fluid source 4 directly to supply pressurized fluid to patient 10 during inhalation and to withhold or reduce the supply of pressurized fluid from patient 10 during exhalation, thereby effectively incorporating the function of pressure regulator 6 into pressurized fluid source 4.

While the use of the diaphragm EMG or effort signal has been described above as the mechanism for triggering the ventilator, it is contemplated that any conventional ventilator triggering technique suitable for use with a spontaneously breathing patient may be used. For example, the pressure and/or flow generated by the patient in patient interface 12 and/or patient circuit 8 can be used to trigger the ventilator. In addition, ventilator 2, and, more specifically, controller 16, can include a timed backup so that if the patient stops breathing for a period of time exceeding a predetermined threshold, the ventilator automatically initiates a breathing cycle.

Pressurized fluid source 4 is, for example, a source of compressed gas, e.g., air, oxygen, helium-oxygen, or other oxygen mixture. The present invention also contemplates that pressurized fluid source is a piston, a bellows or a blower that receives a supply of gas, either from ambient atmosphere or a source of compressed gas, and generates a flow of such gas. Pressure regulator 6 is, for example, a poppet, solenoid, butterfly, rotary, sleeve, or any other valve or valve assembly suitable for use in controlling a flow and/or pressure of fluid delivered to a patient. As noted above controller 16 can control the pressure and/or flow of fluid from pressurized fluid source 4 directly, i.e., without the need for a dedicated pressure control valve, by controlling the speed of the piston, bellows, or blower, thereby effectively combining the functions of pressurized fluid source 4 and pressure regulator 6 as a single unit, as generally indicated by dashed line 27. For present purposes, the combined function of the source of pressurized fluid 4 and a pressure regulator 6 is referred to as a “pressure generator system.” Thus, the pressure generator system includes pressurized fluid source 4 alone, if the flow/pressure of fluid output by the pressurized fluid source can be controlled directly, for example by regulating blower speed, otherwise the pressure generator system includes the combination of source of pressurized fluid 4 and pressure regulator 6.

In one embodiment of the present invention, patient circuit 8 is a single tube or conduit 20 connected between pressure regulator 6 and interface 12, typically referred to as a single-limb circuit. In this embodiment, conduit 20 and/or patient interface 12 includes an exhaust assembly 22 that vents exhaled gases to atmosphere and, thus, represents a known leak in the breathing gas delivery system. An example of a passive exhaust assembly is a hole or slot formed in conduit 20 and/or interface 12 that communicates the interior of the conduit or interface with atmosphere, with no active control over the flow of gas from the system, thereby providing a flow of exhaust gas from the patient circuit and/or interface. The size of the hole is typically selected to be sufficient to purge exhaled gas from the patient circuit. It is to be understood, however, that a wide variety of exhaust devices and configurations are contemplated for use with the ventilator/pressure generator system of the present invention. For example, U.S. Pat. No. 5,685,296 to Zdrojkowski et al. discloses an exhalation device and method where the exhalation flow rate through the device remains substantially constant over a range of pressures in the patient circuit. This exhalation device, which is commonly referred to as a plateau exhalation valve or PEV, is suitable for use with the pressure support system of the present invention.

In another embodiment of the present invention, patient circuit 8 includes a second tube or conduit illustrated by dashed line 24 in FIG. 1, which is typically referred to as a two-limb circuit. Second tube or conduit 24 communicates fluid exhaled by patient 10 to ventilator 2, which includes an active exhaust assembly that monitors and/or controls the venting of exhaust fluids to atmosphere. An example of an active exhaust assembly is a valve that prevents fluid from exhausting to atmosphere when pressurized fluid is supplied to patient 10, i.e., during the inspiratory phase, and that allows gas to escape to atmosphere when the supply of pressurized fluid to patient 10 is terminated or reduced, i.e., during the expiratory phase. Typically, the active exhaust assembly controls the flow of exhaust gas to atmosphere to control the positive end exhalation pressure (“PEEP”) in the patient. Of course, the active exhaust need not be provided in the actuation housing of the ventilator, as generally shown in FIG. 1, but, regardless of its actual location, is typically controlled by or based on signals provided by the ventilator.

The present invention contemplates that patient interface device 12 is any device, either invasive or non-invasive, suitable for communicating a flow of breathing gas from the patient circuit to an airway of the patient. Examples of suitable patient interface devices include a nasal mask, nasal/oral mask, full-face mask, tracheal tube, endotracheal tube, and nasal pillow.

In one embodiment, system 2 may implement process or algorithm 300. In step 302, system 2 may measure or determine ventilation parameters and characteristics of respiratory airflow, such as, for example, the patient's minute ventilation, which is the volume of air exchanged by the patient within a one minute period. When monitoring minute ventilation, an observation window having a time period that ranges between several seconds up to a few minutes may be used to monitor the patient's ventilation. The value may then be scaled proportional to the window length to reflect the amount of air exchanged within a one minute time period. In an embodiment, process 300 monitors minute ventilation once the first breath is completed. In one embodiment, process 300 may use an expanding observation window that begins with the duration of the first breath and that expands out to a maximum of 4 minutes. Using the minute ventilation, a target minute ventilation (MinVol) value may be computed as a fraction of the monitored ventilation. The fractional component may be calculated by a variety of methods. In some embodiments, the patient or clinician may adjust the setting for the percentage or fraction of the minute ventilation. System 2 may also set the percentage or fraction. For example, in one embodiment, the target minute ventilation may be set to 90% of the measured minute ventilation.

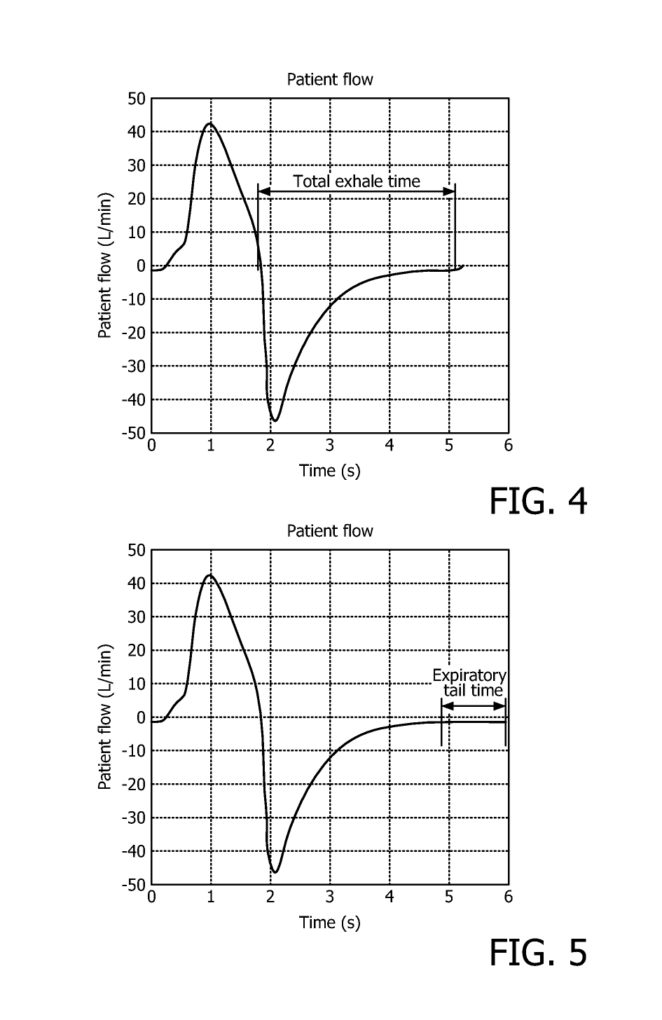

System 2 may also measure or determine the expiratory flow pattern for characteristics of respiratory airflow, such as the expiratory time constant and the expiratory tail time constant. The expiratory tail constant RCe may be calculated as ¼ of the total exhalation time of the patient. The total exhalation time, as shown in FIG. 4, is the time required to completely empty the lungs during passive exhalation. In other words, the exhalation time is the duration of time that the patient continues to have air movement away from the lungs. The expiratory tail time, as shown in FIG. 5, is the time duration after the patient flow has reached 0 liters per minute (1 pm). This is the time period that exists before the next breath begins.

After obtaining values of the parameters, process 300 proceeds to step 304 where a time-based parameter of at least a portion of the patient's respiratory cycle, such as the ideal breath rate, is determined In some embodiments, the ideal breath rate may be determined according to the method as follows.

The system may use the values of the parameters or characteristics obtained in step 302 to determine the time-based parameter of at least a portion of the patient's respiratory cycle, such as the ideal breath rate. The ideal breath rate may be determined according to the following equation (Equation 1.3):

$$f = \frac{\sqrt{1 + 2 a \times RCe \times ( Min Vol - f \times Vd ) / ( Vd )} - 1}{a \times RCe}$$

where:
“a” is a factor that depends on the flow waveform. For sinusoidal flows, a is 2π²/60 or could be optimized based upon patient conditions,
“RCe” is the expiratory time constant,
“MinVol” is the target minute ventilation,
“Vd” is the ventilated dead space, and
“f” is the breath rate frequency

In some embodiments, other measures of ventilation, such as a measure of ventilation longer than one breath, may be used instead of the actual minute ventilation. Accordingly, Equation 1.3 may be re-scaled when other measures of ventilation are used to determine the ideal breath rate.

The ventilated dead space is the volume of air per breath that only reaches portions of the lungs where no beneficial gas exchange occurs. For example, the ventilated dead space may be reflective of the volume of gas that does not reach the respiratory portions, such as the alveoli, but instead remains in the airways (trachea, bronchi, etc.) of the patient. A variety of methods may be used to determine an appropriate ventilated dead space value. In one embodiment, a constant value, such as, for example, 150 ml, may be used, although it is contemplated that other values may be used. In another embodiment, appropriate values may be fit into a table referenced against the monitored minute ventilation. In some embodiments, the values in the table may be reflective of a relationship between the size (e.g., body weight) of the patient and the ventilated dead space values. For example, higher minute ventilation values may be associated with larger patients, and thus such patients may have larger ventilated dead space values.

Equation 1.3 may be iteratively run by inserting the calculated breath rate f back into the equation until f does not change significantly between cycles. In one embodiment, Equation 1.3 is iteratively run by inserting the calculated breath rate f back into the equation until the difference between the values off between cycles is less than 0.5 breaths per minute. The initial value of f may be the current breath rate.

In one embodiment, when the RCe is 0.5 seconds, the following table is generated to show the minute ventilation and the resulting ideal breath rate f:

| Target Minute Ventilation | f ideal |
|---|---|
| 5 | 11 |
| 6 | 13 |
| 7 | 14 |
| 8 | 16 |
| 9 | 17 |
| 10 | 18 |

Alternatively or additionally, system 2 may use the following method to calculate ideal breath rate. In some situations, the exhaled flow of the sleeping patient might not be available. This may occur when the sleeping patient inhales through his nasal passages but exhales orally. For this method, the target minute ventilation and the ventilated dead space Vd parameters may be used to calculate the ideal breath rate. This rate may be calculated when the ratio of ventilated dead space to total ventilation is held to a constant value, such as, for example 30%, which is an acceptable number in clinical practice.

Using this second method, the dead space minute ventilation and total minute ventilation (also known as the target minute ventilation) may be such that:

Dead Space Minute Ventilation/Target Minute Ventilation=30% where Dead Space Minute Ventilation=Volume Dead Space*f

As mentioned above, the volume dead space, or the ventilated dead space, may have a value of 150 ml. As such, the ideal breath rate f may be determined using the following equation (Equation 1.4):

Ideal Breath Rate f=0.3*(Target Minute Ventilation)/0.150

By inserting target minute ventilation values obtained in step 302 into Equation 1.4, the following values may be obtained for the ideal breath rate f:

| Target Minute | |
|---|---|
| Ventilation | f ideal breath rate |
| 5 | 10 |
| 6 | 12 |
| 7 | 14 |
| 8 | 16 |
| 9 | 18 |
| 10 | 20 |

As mentioned above, other measures of ventilation may be used instead of actual minute ventilation, such as a measure of ventilation longer than one breath. Therefore, Equation 1.4 may also be re-scaled when other measures of ventilation are used.

The ideal breath rate may be calculated using one or both of the methods described above. In embodiments that use both methods to calculate the ideal breath rate, the results may be compared against each other to determine if the results are consistent. The results may be averaged in some embodiments. In some embodiments, one method may be used as the primary method and the other as an alternative method in the event that not all of the input parameters are available.

Process 300 may then proceed to step 306 after the ideal breath rate f has been obtained. The target tidal volume may be determined using the following equation:

Target Tidal Volume=Target Minute Volume/f

where:
“f” is the ideal breath rate, and

### Target Minute Volume=Target Minute Ventilation.

As mentioned above, the target minute volume or ventilation may be obtained in step 302 when system 2 monitors the patient's ventilation parameters. Using the ideal breath rate f calculated via the first method described above in step 304 and the following minute ventilation values, the following target tidal volume may be obtained:

| Minute Ventilation | f ideal | Tidal Volume Target |
|---|---|---|
| 5 | 11 | 455 |
| 6 | 13 | 462 |
| 7 | 14 | 500 |
| 8 | 16 | 500 |
| 9 | 17 | 529 |
| 10 | 18 | 555 |

In some embodiments, other time-based parameters of at least a portion of the patient's respiratory cycle may be used to determine the target tidal volume. For example, in one embodiment, an inspiratory time may be used to determine the target tidal volume. In some embodiments, other target breath-amplitude-based parameter, such as target peak flow may be used instead of or in addition to the target tidal volume. In another embodiment, mean inspiratory flow may be used instead of or in addition to the target tidal volume.

It is contemplated that in some embodiments, system 2 may measure or determine respiratory resistance of the patient. Alternatively or additionally, system 2 may also measure or determine lung compliance of the patient. Thus, any one or any combination of these and other characteristics may be used when determining the target tidal volume to be delivered to the patient. The respiratory resistance of the patient and the lung compliance of the patient may be determined using various methods, such as, for example, those described in U.S. Pat. No. 5,884,622 and U.S. patent application Publication No. 2006/0249148.

In some embodiments, process 300 may then proceed to step 308. Alternatively, the process may skip this step 308 and proceed to step 310. In this step 308, system 2 monitors the parameters of patient 10 and protects the patient against auto-peep. Auto-peep occurs when the next breath commences before the last breath finishes. Auto-PEEP is caused by gas trapped in the alveoli at the end of expiration. This gas is not in equilibrium with the atmosphere and exerts a positive pressure, thereby increasing the work of breathing. In this step 308, an analysis is performed to ensure that auto peep does not occur and that the patient 10 has time to fully exhaust the inspired volume before the next breath is to occur. For a given breath rate and tidal volume, limits are determined to ensure that some expiratory tail time exists or that the expiratory tail time is above a certain threshold, such as, for example, 0.5 seconds. The threshold may vary, and may be any number above 0 seconds. The expiratory tail time is monitored as discussed above in step 302 to ensure that the values of the Tidal Volume, RCe and breath rate are such that they provide ample exhalation time. The target tidal volume and breath rate may be modified as a result of this step. In one embodiment, for each breath that is delivered, it is expected that at least 0.5 seconds of expiratory tail time exist before the next breath is delivered. If no tail time exists or is below the threshold, the target tidal volume is reduced until expiratory tail time does exist or is above the threshold.

Process 300 proceeds to step 310 where system 2 provides the target tidal volume to the patient 10. System 2 may deliver closed loop pressure support to patient 10 using the target tidal volume as the setpoint. A simple closed loop control loop may be utilized to deliver a target tidal volume to the patient, as described in U.S. Pat. No. 7,011,091. The target volume may be delivered to the patient 10 using pressure support, which will be described in more detail later.

When providing the target tidal volume, the system may determine amount of volume of breathing gas that is actually being delivered to the patient or received by the patient 10. System 2 may determine this amount by using the methods described in U.S. Pat. No. 7,011,091. In some embodiments, determining the average volume over multiple breath cycles requires determining the volume of breathing gas delivered during each breath. This may be accomplished relatively easily in a two-limb patient circuit because the ventilator controls the amount of fluid exhausted to atmosphere. In addition, there is considered to be substantially no leak from the patient circuit in a two-limb configuration. Therefore, the total volume of breathing gas delivered to the patient 10 during each breath is determined using any conventional technique, such by providing a flow meter in the exhaust limb to measure the flow rate of exhaust gas and, from this, determine the volume of gas exhausted during each breath cycle.

In a single limb circuit, however, determining the volume of breathing gas actually delivered to or received by the patient 10 during each breath is more difficult due to the fact that there is a relatively large intentional leak in the patient circuit and potential unintentional leaks at the interface between the patient and the interface device. U.S. Pat. No. 5,148,802 to Sanders et al., U.S. Pat. No. 5,313,937 to Zdrojkowski et al., U.S. Pat. No. 5,433,193 to Sanders et al., U.S. Pat. No. 5,632,269 to Zdrojkowski et al., and U.S. Pat. No. 5,803,065 to Zdrojkowski et al., the contents of each of which are incorporated by reference into the present invention, describe techniques for detecting and estimating leak and managing the delivery of breathing gas to the patient in the presence of leaks. A brief description of this process is provided below.

In a single limb circuit, the volume of fluid received patient 10 over a breath cycle is determined from a difference between the volume of fluid supplied to the patient by the ventilator, i.e., the volume of fluid output by the ventilator, and the volume of fluid leaking from the ventilator system, which includes leak from the patient circuit and leak from the patient interface device, during that breath cycle. Typically, most of the leak is from the exhaust vent in the patient circuit. More specifically, fluid leaking to atmosphere is generally the result of a known leak, such as the exhaust flow provided by exhaust assembly 22 in the single-limb circuit, and unknown leaks, such as a leak at the interface between the patient and patient interface device 12. The volume of fluid received by patient 10 at any given time may be estimated according to the methods set forth in U.S. Pat. No. 7,011,091, the contents of which are incorporated by reference herein in its entirety. In a single limb system, system 2 may account for the leak rate when estimating the volume of fluid delivered to a patient during a breath cycle. The estimated amount of volume that is received by the patient may be used to determine the amount of pressure that should be generated by the pressure generator system to provide the target tidal volume.

In some embodiments, adjusting a volume of fluid supplied to the patient is accomplished by controller 16 causing pressure regulator 6 to adjust the inspiratory positive airway pressure (IPAP) level.

In some embodiments, the pressure support level may be adjusted. As understood by those skilled in the art, the term “pressure support” is defined as the difference between the inspiratory positive airway pressure and the expiratory positive airway pressure. Stated mathematically, pressure support (PS) is defined as follows: PS=IPAP−EPAP. Thus, adjusting the pressure support is accomplished by adjusting the IPAP and/or the EPAP level.

In one embodiment, the present invention adjusts the IPAP level, which is referred to as IPAP, from one inspiratory phase to the next so that the average volume over multiple breaths corresponds to a target volume. It is believed that by varying the IPAP level for the delivery of breathing gas to the patient so as to achieve an average volume over multiple breaths, rather than a target volume for each breath as done in the VTV or VAPS mode, for example, the ventilation mode of the present invention is more comfortable for the spontaneously breathing patient while still being responsive to the patient's changing respiratory demands.

When pressurized fluid is supplied to patient 10 during first breath cycle 30, pressure regulator 6 sets the inspiratory positive airway pressure level for the fluid delivered to the patient. The IPAP level is may be set between a maximum IPAP, IPAPₘₐₓ, and a minimum IPAP, IPAPₘᵢₙ. IPAP. and IPAPₘᵢₙ are typically set by the clinician. However, the present invention also contemplates that IPAPₘₐₓ, IPAPₘᵢₙ or both can be automatically set by the ventilator. For example, once the user sets IPAPₘᵢₙ, the ventilator can set IPAPₘₐₓ automatically as a fixed percentage of fixed pressure above IPAPₘᵢₙ. IPAPₘᵢₙ can be set in a similar fashion after the clinician sets IPAPₘₐₓ.

System 2 may also monitor the amount of time that has elapsed since the last transition from the expiratory to the inspiratory phase of the respiratory cycle. If no spontaneous inspiratory effort is detected over a certain period of time, a “machine triggered breath” may be automatically delivered to the patient by system 2, thus ventilating the lungs. In some embodiments, a machine triggered breath is delivered to the patient if the patient fails to initiate a spontaneous breath within the time associated with T_{period}=60/fₘᵢₙ where Fmin may be equal to f the ideal breath rate, or may be 2 breaths per minute less than f the ideal breath rate or may be a fixed value of 10 breaths per minute.

Controller 16 may provide the calculated target tidal volume to the patient according to the methods set forth in U.S. Pat. No. 7,011,091, the contents of which are incorporated by reference herein in its entirety. In one embodiment, controller 16 (a) determines, for each inspiratory phase of a respiratory cycle of the patient, a volume of fluid received by the patient based on the parameter indicative of a volume of fluid delivered to the patient provided by the sensor, (b) determines an average volume of fluid received by the patient over a plurality of inspiratory phases, (c) compares the average volume of fluid received by the patient to the target tidal volume determined using the methods described above, and (d) causes the pressure generator system to adjust the pressure or the rate of flow of fluid output thereby based on this comparison. In response to activating the ventilator 2, the controller 16 may cause pressurized fluid to be supplied to patient 10 during the inspiratory phase and cause the flow of pressurized fluid to be reduced or withheld from the patient during exhalation. As noted above, a target tidal volume is determined by system 2, which is a desired volume of fluid to be delivered to the patient during each respiratory cycle.

In one embodiment, when providing a target tidal volume to the patient, the controller 64 may determine a pressure support error, which represents an estimate of how much pressure support should be added or removed from the current pressure support level of the patient, as described in U.S. Pat. No. 7,011,091. In such embodiments, the controller 64 may change the pressure support level gradually over several minutes so as to move the pressure support error toward zero. In an embodiment, the EPAP pressure remains fixed, and the IPAP pressure is ramped over several minutes. The ramp time is limited by the use of a one minute averaging array. The array may be filled with new data before the decision to increase or decrease the pressure can be made again so prevent controlling the system faster than the measurement system operates.

In one embodiment, the controllers may determine whether the tidal volume each breath or respiratory cycle of the patient is within normal parameters. If so, the system may use the data associated with that breath in the further processing steps. If the breath data is not within normal parameters, the data associated with that breath is thrown away. The present invention also contemplates monitoring the number of breaths that are discarded or considered not normal, so that an alarm can be indicated, for example, if too many non-normal breaths are detected. Such an occurrence may indicate that the patient is experiencing a pronounced period of respiratory disorder, that the pressure support system is not functioning properly, or both.

It should be noted that the present invention contemplates determining the exhaled tidal volume in an invasive or a non-invasive ventilatory system using the methods discussed above with respect to determining the inhaled tidal volume. That is, the same principles discussed above with respect to determining the inhaled tidal volume apply to determining the exhaled tidal volume. Of course, any conventional technique can be used to determine the expiratory tidal volume.

Based on the foregoing, it can be appreciated that the present invention provides an apparatus and method for adjusting the volume of fluid supplied to a patient during a breathing cycle as a function of the volume of fluid received by the patient during a previous breathing cycle, thereby avoiding patient discomfort. This allows the patient to alter his or her breathing pattern temporality, such as by taking a very shallow or a very deep breath, during one breathing cycle without the ventilator overreacting to these minor variations.

In some embodiments, ventilator 2 may provide ventilation therapy by only providing target tidal volume. That is, the ventilator may provide the target tidal volume as the entire ventilation therapy. In some embodiments, the ventilator 2 may provide additional forms of therapy in addition to providing target tidal volume. That is, the target volume delivery may only be one component of the multiple component ventilation therapy. For example, in some embodiments, the ventilator 2 may also be in the form of an auto servo ventilator, which is described in U.S. patent application No. 2006/0070624, the contents of which are incorporated by reference herein in its entirety. In such embodiments, the tidal volume calculation as described above may be used as a minimum tidal volume to calculate a baseline pressure support value or a minimum IPAP value. Ventilator 2, using an auto servo algorithm, may then adjust the pressure support value to provide stable breathing. In some embodiments, the ventilator may increase or decrease the inspiratory pressure to adjust the pressure support value. Thus, the baseline pressure support value calculated according to the target tidal volume may be increased or decreased depending on the adjustment by the ventilator 2 based on the auto servo algorithm to achieve a modified pressure support value to be delivered to the patient. It is contemplated that a third algorithm, such as flex described in U.S. Pat. No. 6,532,956, may be added to produce a summed result for the pressure support provided to the patient.

Embodiments of the invention, such as the controller, microprocessors, or processors, for example, may be made in hardware, firmware, software, or various combinations thereof. The invention may also be implemented as instructions stored on a machine-readable medium, which may be read and executed using one or more processing devices. In one embodiment, the machine-readable medium may include various mechanisms for storing and/or transmitting information in a form that may be read by a machine (e.g., a computing device). For example, a machine-readable storage medium may include read only memory, random access memory, magnetic disk storage media, optical storage media, flash memory devices, and other media for storing information, and a machine-readable transmission media may include forms of propagated signals, including carrier waves, infrared signals, digital signals, and other media for transmitting information. While firmware, software, routines, or instructions may be described in the above disclosure in terms of specific exemplary aspects and embodiments performing certain actions, it will be apparent that such descriptions are merely for the sake of convenience and that such actions in fact result from computing devices, processing devices, processors, controllers, or other devices or machines executing the firmware, software, routines, or instructions.

It can be appreciated that the embodiments are not to be limited to the specific time periods, percentages, and constants noted above. Rather, other values for these quantities can be used so long as the general principles of the present invention are maintained. In addition, these quantities need not be fixed. Instead, they can be dynamically altered by the controller 64 based on the monitored condition of the patient. This can be done, for example, to treat the patient more aggressively if they are not responding to the current treatment scheme, and vise versa.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A system for controlling and regulating breathing gas supplied to a patient, comprising:
a pressure generator system adapted to provide breathing gas to a patient;
a patent circuit operatively coupled to the pressure generator system to deliver the flow of breathing gas to the patient;
an interface device operatively coupled to the patient circuit to communicate the flow of breathing gas to an airway of the patient;
a sensor operatively coupled to one or more of the pressure generator system, the patient circuit, and the interface device, the sensor operable to detect one or more parameters indicative of a flow of breathing gas delivered to the patient; and
a controller operatively coupled to the sensor and the pressure generator system, wherein the controller determines at least one characteristic of respiratory airflow of a patient, and, based on the at least one characteristic of respiratory airflow, of the patient, calculates a target time-based parameter of at least a portion of the patient's respiratory cycle, and calculates a target breath-amplitude-based parameter to be delivered to the patient, wherein the at least one characteristic of respiratory airflow of the patient comprises a target minute ventilation, and wherein the controller calculates an ideal breath rate by using a value of a ventilated dead space and the target minute ventilation value.

2-3. (canceled)

4. The system of claim 1, wherein the ideal breath rate is calculated using a ratio of the value of ventilated dead space to the target minute ventilation value.

5. The system of claim 4, wherein the ratio is equivalent to 0.3.

6. The system of claim 1, wherein the target minute ventilation is calculated as a fraction or percentage of an actual minute ventilation of the patient.

7. The system of claim 1, wherein the at least one characteristic of respiratory airflow of the patient comprises a measure of patient ventilation over a course of time longer than one breath.

8. The system of claim 1, wherein the target time-based parameter comprises ideal breath rate or ideal inspiratory time.

9. The system of claim 8, wherein the controller determines the ideal breath rate by iteratively calculating the ideal breath rates until the difference between the calculated ideal breath rates in each iteration is less than a certain threshold amount.

10. (canceled)

11. The system of claim 1, wherein the target breath-amplitude-based parameter comprises tidal volume, peak flow, or mean inspiratory flow.

12. The system of claim 1, wherein the at least one characteristic of respiratory airflow of the patient comprises an exhalation time of the patient, a respiratory resistance of the patient, or a lung compliance of the patient.

13. The system of claim 1, wherein the at least one characteristic of respiratory airflow of the patient comprises an exhalation time of the patient, wherein the controller determines an expiratory time constant, and wherein the expiratory time constant is a fraction or percentage of the exhalation time used to calculate the time-based parameter.

14-17. (canceled)

18. The system of claim 1, wherein an actual minute ventilation is used to calculate a target minute ventilation value.

19. The system of claim 1, wherein the controller calculates an ideal breath rate using a value reflective of a ventilated dead space.

20. The system of claim 1, wherein the at least one characteristic of respiratory airflow comprises an expiratory tail time, and wherein the controller adjusts the target tidal volume so that the expiratory tail time is above a certain threshold amount.

21. The system of claim 20, wherein the threshold amount is 0.5 seconds.

22-44. (canceled)

45. A system for controlling and regulating breathing gas supplied to a patient, comprising:
means for determining at least one characteristic of respiratory airflow of a patient, wherein the at least one characteristic of respiratory airflow of the patient comprises a target minute ventilation;
means for calculating a target time-based parameter of at least a portion of the patient's respiratory cycle based on the at least one characteristic of respiratory airflow of the patient;
means for calculating a target breath-amplitude-based parameter based on the at least one characteristic of respiratory airflow of the patient; and
means for calculating an ideal breath rate by using a value of a ventilated dead space and the target minute ventilation value.
